(19) United States
(12) Patent Application Publication
(10) Pub. No.: US 2026/0232479 A1
(43) Pub. Date: Aug. 13, 2026

(54) HYDROGEL FOR OSTOMY APPLIANCES

(71) Applicant: Hollister Incorporated, Libertyville, IL (US)

(72) Inventors: Sohaila Abdelhamid, Chicago, IL (US); Mallory Brodnik, Fairfax, VA (US); Riley Forst, Chicago, IL (US); Stephanie Nofz, Montreal (CA); Lingfeng Tang, Guangzhou (CN); Luke Teasley, Fairfax Station, VA (US); Mustafa Guler, Wilmette, IL (US); Terry Johnson, Chicago, IL (US); Adrian Defante, Chicago, IL (US); Abram Janis, Grayslake, IL (US)

(73) Assignee: Hollister Incorporated, Libertyville, IL (US)

(21) Appl. No.: 19/153,056

(22) PCT Filed: Mar. 7, 2024
(86) PCT No.: PCT/US2024/018796

§ 371 Date: Aug. 1, 2025

Related U.S. Application Data
(60) Provisional application No. 63/489,312, filed on Mar. 9, 2023.

(51) Int. Cl. A61F 5/443; A61F 5/44

(52) U.S. Cl. CPC A61F 5/443; A61F 5/4404

(57) ABSTRACT

An ostomy skin barrier is formed from a double network hydrogel (DN hydrogel) comprising at least two different polymer networks, such as a polyacrylamide network, a sodium alginate network, and chitosan network. The DN hydrogel can also include a pH biosensor configured to change color when exposed to stoma dejecta to indicate a leakage.

### BACKGROUND

The present disclosure relates to ostomy skin barriers, and more particularly to hydrogels for ostomy skin barriers.

Ostomy appliances, such as an ostomy pouch, are used to collect stoma dejecta (fecal material and/or urine) for patients with a stoma created by a surgery (e.g., colostomy, ileostomy or urostomy). An ostomy appliance is typically attached to user's peristomal skin via an ostomy wafer or an ostomy face plate including a skin barrier. A skin barrier is designed to securely attach and support an ostomy appliance, while absorbing moisture and stoma dejecta (for example, from a leakage) from the peristomal skin and facilitate peristomal skin health. Some ostomy patients also use an ostomy ring that is made using a skin barrier material to fill in uneven skin contours around the stoma and/or to create a flatter peristomal skin surface for improved attachment of an ostomy wafer.

When an ostomy wafer is not properly fitted to the user, a leakage can occur and allow stoma dejecta to come in contact with the skin surrounding the stoma. This can cause irritation and can lead to broken skin and infection as well as interference with the adhesion of a skin barrier to user's skin. Many known ostomy skin barriers are formed of hydrocolloid adhesives, which can break down when exposed to stoma dejecta. Further, due to the continuous and prolonged use of ostomy appliances, user's peristomal skin is always at risk of irritation and injuries.

Hydrogels are biomaterials designed for medical applications, such as drug delivery, regenerative medicine, tissue adhesion and wound treatments. Hydrogels are typically mechanically weak and have relatively low elastic moduli, which have historically presented limitations in applications requiring higher mechanical properties, such as ostomy skin barriers.

The present disclosure provides improved ostomy skin barriers that can reduce a risk of peristomal skin irritation and injuries, while improving user's comfort and peristomal skin health.

### BRIEF SUMMARY

Hydrogels for ostomy skin barrier appliances are provided according to various embodiments.

In one aspect, an ostomy skin barrier may be formed from a double network hydrogel (DN hydrogel) comprising at least two different polymer networks. For example, the DN hydrogel may comprise a polyacrylamide network, a sodium alginate network, and a chitosan network. In such an embodiment, the chitosan network may interact with user's skin when the ostomy skin barrier is attached to the user's skin to facilitate adhesion between the DN hydrogel and the user's skin.

In an embodiment, the DN hydrogel may be formed from a mixture comprising sodium alginate, acrylamide (AAm), calcium sulphate (CaSO₄), ammonium persulfate (APS), N,N′-methylenebisacrylamide (MBAA), and tetramethylethylenediamine (TEMED). The mixture may be crosslinked via chemical or physical means, such as light (e.g. UV or visible), temperature, and/or mechanical stimulation.

In some embodiments, the DN hydrogel may also comprise a pH biosensor. The pH biosensor may be configured to change color when exposed to stoma dejecta to indicate a leakage.

In an embodiment, the pH biosensor may be a universal pH indicator configured to change color along a range of pH from about 1 to about 14. For example, the universal pH indicator may be formed from a mixture of Methyl Red, Phenolphthalein, Ethanol, Bromothymol Blue, and NaOH. In another embodiment, the pH biosensor may be a pH indicator configured to change color along a range of pH from about 4 to about 10. In such embodiments, the pH biosensor may be configured to indicate a pH level of stoma dejecta that comes in contact with the ostomy skin barrier, wherein a color of the skin barrier may be compared to a pH color scale for determination of the pH level of the stomal dejecta.

In an embodiment, the DN hydrogel may be formed by adding the pH biosensor to an alginate/AAm solution prepared by dissolving sodium alginate and AAm in diH2O, and mixing MBAA, APS, CaSO4, and TEMED into the alginate/AAm solution to form a gel, and curing the gel using UV light.

In some embodiments, the DN hydrogel may be configured to swell in response to a change in pH. In an embodiment, the DN hydrogel may be a basic DN hydrogel configured to swell when exposed to stoma dejecta having an acidic pH. In another embodiment, the DN hydrogel may be an acidic DN hydrogel configured to swell when exposed to stoma dejecta having a basic pH. In yet another embodiment, the DN hydrogel may be an amphiphilic hydrogel configured to swell when exposed to stoma dejecta having an acidic pH or a basic pH.

In another aspect, an ostomy appliance for securing an ostomy pouch to a user may comprise the ostomy skin barrier formed according to any one of the foregoing embodiments. In an embodiment, the ostomy appliance may be an ostomy wafer attached to an ostomy pouch and configured to support the ostomy pouch when attached to the user. In another embodiment, the ostomy appliance may be an ostomy faceplate comprising a body-side coupling member, wherein the ostomy pouch may include a pouch-side coupling member configured to engage with the body-side coupling member to attach the ostomy pouch to the ostomy faceplate. In such an embodiment, the ostomy faceplate may be configured to support the ostomy pouch when attached to the user.

In yet another aspect, an ostomy ring may be formed from the ostomy skin barrier formed according to any one of the foregoing embodiments. The ostomy ring may be configured to work with an ostomy wafer or an ostomy faceplate to securely attach the ostomy wafer or the ostomy faceplate to a user.

The DN hydrogel of any of foregoing embodiments may be configured to have viscoelastic properties similar to that of user's skin. For example, the DN hydrogel may be configured to have an elastic modulus of about 10 kPa to about 1 MPa.

The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1A is a schematic illustration of a prior art skin barrier formed from a hydrocolloid adhesive attached to user's skin;

FIG. 1B is a schematic illustration of a skin barrier formed from a chitosan hydrogel adhesive according to an embodiment;

FIG. 2 is a graph showing volume of hydrogels vs pH;

FIG. 3 is a schematic cross sectional view of an ostomy appliance comprising an ostomy pouch and an ostomy wafer according to an embodiment;

FIG. 4 is a schematic cross sectional view of the ostomy wafer of FIG. 3; and

FIG. 5 is an illustration of an ostomy ring according to an embodiment.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular. The words “first,” “second,” “third,” and the like may be used in the present disclosure to describe various information, such information should not be limited to these words. These words are only used to distinguish one category of information from another. The directional words “top,” “bottom,” up,“ ”down,“ front,” “back,” and the like are used for purposes of illustration and as such, are not limiting. Depending on the context, the word “if” as used herein may be interpreted as “when” or “upon” or “in response to determining.”

Each ostomate's stoma and peristomal skin topography is unique. The complexity and variations in stoma and peristomal skin topographies among ostomates present great challenges in providing skin barrier appliances that properly fit user's peristomal topography. As such, ostomates often experience a leakage, which exposes the skin barrier and the peristomal skin to stoma dejecta. When expose to stoma dejecta, skin complications, such as moisture-associated skin damage, irritant contact dermatitis and infections, can develop. Skin barriers formed from a hydrocolloid adhesive can breakdown when exposed to stoma dejecta for an extended period. Further, hydrocolloid adhesives typically have a significantly greater elastic modulus when compared to abdominal skin. Thus, skin barriers formed from such hydrocolloid adhesives can be uncomfortable for users.

Hydrogels are water-insoluble, three-dimensional network of polymer chains capable of holding large amounts of liquid. In general, hydrogels can fall into two major categories: chemical hydrogels and physical hydrogels. Chemical hydrogels have covalent cross-linking bonds, whereas physical hydrogels have non-covalent bonds. Hydrogels may be designed for use in or on the human body and can provide excellent biocompatibility. Hydrogels typically have a significantly lower elastic modulus when compared to hydrocolloid adhesives and can be configured to have a similar elastic modulus to that of abdominal skin to improve user's comfort. However, most hydrogels are relatively weak and do not have mechanical properties suitable for ostomy skin barriers for supporting an ostomy appliance.

Double network hydrogels (DN hydrogels) comprising at least two different polymer networks, such as those formed from poly(2-acrylamido-2-methylpropanesulfonic acid) (PAMPS) and polyacrylamide (PAAm), can be configured to have improved mechanical properties including fracture toughness of 102-103 J/m², fracture tensile stress of 1-10 MPa, and fracture tensile strain of 1000-2000%. Such improvements in mechanical properties of the DN hydrogels may be attributed to their unique network structure as well as the entanglements within the structure. Hybrid DN hydrogels include both physically crosslinked components and chemically crosslinked components, wherein the first network is formed by physically crosslinked gels and the second network is chemically crosslinked. The associations between the networks may be non-covalent interactions such as van der Waals interactions, hydrogen bonds, and electrostatic association, depending on the intrinsic properties of the polymers.

An ostomy skin barrier may comprise a DN hydrogel (the term “DN hydrogel” herein broadly includes both DN hydrogel and hybrid DN hydrogel) according to various embodiments. The skin barrier comprising a DN hydrogel may be configured to provide improved biocompatibility, non-volatility, and superior flexibility (low elastic modulus) when compared to skin barriers formed from hydrocolloid adhesives. Further, the skin barrier comprising a DN hydrogel may be configured to provide adhesive properties suitable for supporting ostomy appliances and have antimicrobial properties to reduce the risk of peristomal skin infections and complications.

In an embodiment, an ostomy skin barrier may comprise a hybrid DN hydrogel. The hybrid DN hydrogel may include a physically crosslinked alginate component and a chemically crosslinked acrylamide (AAm) component. In some embodiments, the hybrid DN hydrogel may also comprise chitosan (also referred to herein as “chitosan hydrogel adhesive”). In an embodiment, the chitosn hydrogel adhesive may comprise a polyacrylamide network, sodium alginate network, and chitosan network.

In an embodiment, the chitosan hydrogel adhesive may be formed from a mixture of sodium alginate, AAm, CaSO4, ammonium persulfate (APS), N,N′-methylenebisacrylamide (MBAA), and tetramethylethylenediamine (TEMED). A sample of the chitosan hydrogel adhesive was formed via the following synthesis steps: 1) 1.028 g of sodium alginate and 5.3575 g of AAm were dissolved in 50 ml of diH₂O for 24 hours until the sodium alginate was dissolved; 2) 17.5 ml of the alginate/AAm solution was mixed with 600 μl of MBAA (0.2 g per 100 ml), 100 μl of APS (0.75 M), 400 μl of CaSO4 (0.27 M) and 10 μl of TEMED; and 3) the solution was quickly poured into a mold to prevent quick gelation of the alginate with the ionic crosslinker, and the gel was cured under UV light.

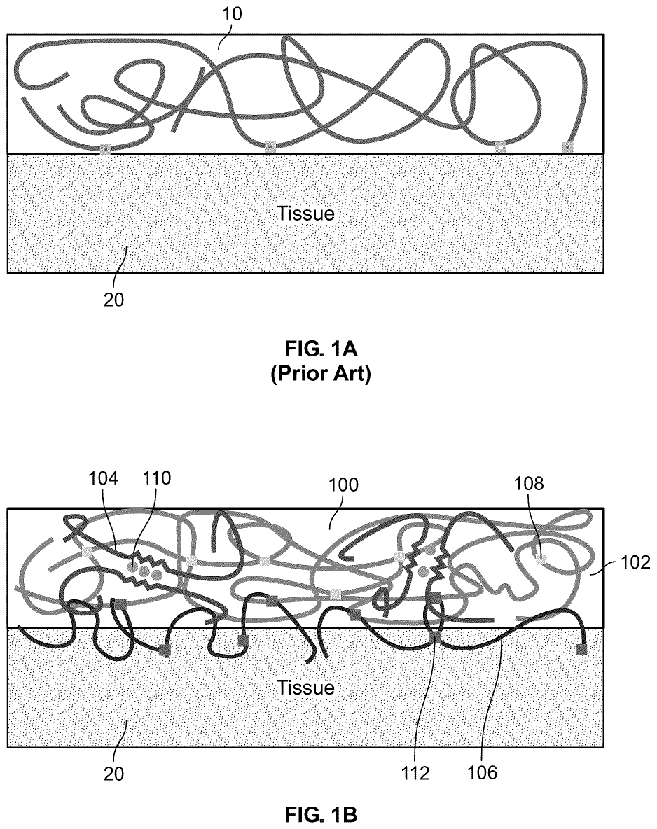

In an embodiment, a skin barrier is formed using the chitosan hydrogel adhesive. In such an embodiment, the chitosan network in the chitosan hydrogel adhesive may function as an adhesion enhancer to provide a strong and flexible topological adhesion between user's skin and the chitosan hydrogel adhesive. FIG. 1A is a schematic illustration of a prior art skin barrier 10 formed from a hydrocolloid adhesive attached to user's skin 20. FIG. 1B is a schematic illustration of a skin barrier 100 formed from a chitosan hydrogel adhesive attached to user's skin 20 according to an embodiment. The skin barrier 100 may be formed from a chitosn hydrogel adhesive comprising polyacrylamide network 102, sodium alginate network 104, chitosan network 106, polyacrylamide crosslinker 108, ionic crosslinker (calcium ion) 110, and chitosan crosslinker 112. As shown in FIG. 1B, the chitosan network 106 may interact with user's skin 20 and function as an adhesion enhancer to provide the adhesive properties sufficient for supporting an ostomy appliance, such as an ostomy pouch.

In some embodiments, an ostomy skin barrier may be formed from a DN hydrogel configured to respond to a change in a pH level.

The pH of urine in a healthy human ranges from 4.5-8.0. The pH of a healthy human's feces is typically about 6.6 and sits slightly acidic as a result of the fermentation of sugars and the production of fatty acids in the human digestive system. In ileostomy patients, the median pH is 7.0 in duodenum, 6.3 in the proximal region, and 7.3 in the distal part of the small intestine slightly more basic than people without ileostomy procedures. There exist numerous links between the pH of fecal matter and urine, and diseases, specifically colonic cancers. It is well documented that beneficial bacteria tend to prefer slightly acidic gut microbiomes whereas harmful bacteria prefer a more basic environment. The proton exchange that occurs in many biochemical reactions conducted by bacteria serve as a method with which they alter and control the pH of their environment. As such, the population of harmful bacteria grows as that of beneficial bacteria decreases. Further, the gastrointestinal pH and the effect it has on the bacterial population can impact absorption of vitamins, electrolytes, and activities of digestive enzymes. Studies have shown that there is a significantly increased correlation between mortality and bacteremia percentages in patients with an overly acidic or basic fecal matter.

Research regarding pH of feces and the gastrointestinal tract has illuminated the relationship between colorectal cancer and fecal pH. It has been shown that a high colonic pH can promote carcinogen creation from bile acids in turn leading to colorectal cancer initiating and progressing in intestinal environments of a higher pH. Population studies have shown that patients without colon cancer exhibit a fecal pH of 6.6±0.44 whereas patients with colon cancer have a fecal pH of 8.0±0.44 (with a p value <0.01). Thus, it is desirable to monitor pH of ostomates' stoma dejecta.

In an embodiment, the ostomy skin barrier may be formed from a DN hydrogel comprising a pH biosensor configured to indicate a pH level of the skin barrier and change color in response to a change in pH. For example, the skin barrier may change color when exposed to stoma dejecta. In such an embodiment, the skin barrier may be used to monitor user's stoma dejecta pH. The pH biosensor may be configured to provide a distinct visual cue, such as color change, when exposed to stoma dejecta to indicate a leakage. In response to the visual cue or color change, a user may change the skin barrier to prevent propagation of leakage and further exposure to stoma dejecta and avoid skin complications caused by irritants in the dejecta.

In an embodiment, the ostomy skin barrier may be formed from a DN hydrogel comprising a pH indicator configured to change color along the full pH range, i.e., pH 1-pH 14 (“universal pH indicator). In such an embodiment, a user may be provided with a pH color scale for comparing the color of the skin barrier to determine the pH level. The universal pH indicator may be formed from a mixture of Methyl Red, Phenolphthalein, 95% Ethanol Solution, Bromothymol Blue, and 0.1 M NaOH.

In another embodiment, the ostomy skin barrier may be formed from a DN hydrogel comprising a pH indicator configured to change color only along a range of pH from about 4 to about 10 as detection of extreme pH levels may not be necessary when dealing with fecal or dejecta pH.

In an embodiment, the DN hydrogel comprising a pH indicator may be formed by adding the pH indicator to an alginate/AAm solution prepared by dissolving sodium alginate and AAm in diH₂O, and mixing MBAA, APS, CaSO4, and TEMED into the alginate/AAm solution to form a gel, and curing the gel using UV light.

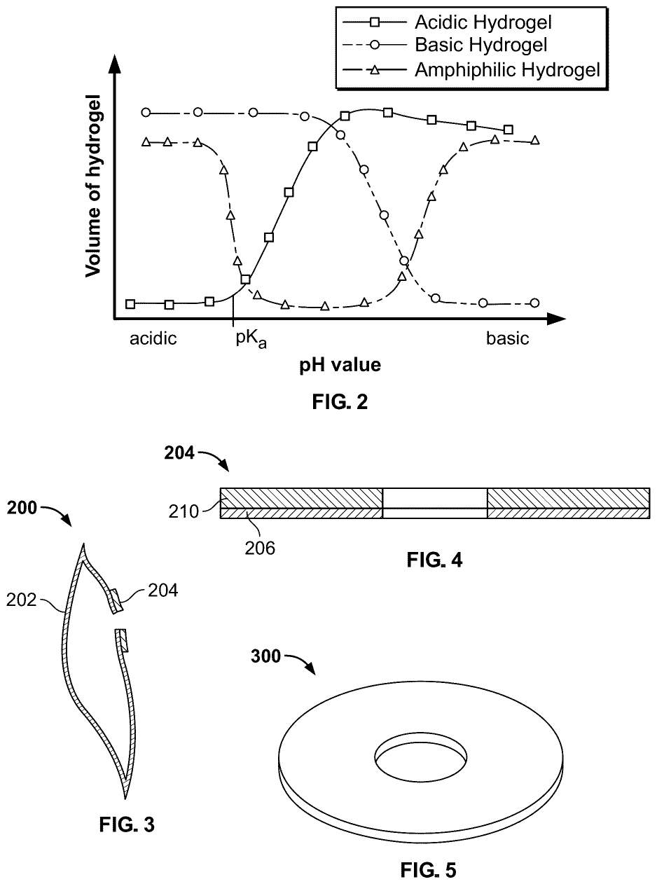

In an embodiment, the ostomy skin barrier may be formed from a DN hydrogel configured to swell in response to a change in pH. For example, the ostomy skin barrier may be formed from an acidic DN hydrogel that swells when exposed to stoma dejecta having a basic pH. In another example, the ostomy skin barrier may be formed from a basic DN hydrogel that swells when exposed to stoma dejecta having an acidic pH. In yet another example, the ostomy skin barrier may be formed from an amphiphilic hydrogel that swells when exposed to dejecta having an acidic pH or a basic pH. FIG. 2 shows a graph of volumes of acidic hydrogel, basic hydrogel and amphiphilic hydrogel vs pH of environment. In such embodiments, the swelling of the DN hydrogel may serve to increase the surface area and/or volume of a skin barrier when a leak occurs and prevent or minimize propagation of the breach and resultant leakage of stoma dejecta.

In any of the foregoing embodiments, the DN hydrogel may be configured to have viscoelastic properties similar to that of skin to provide a skin barrier that bends and folds with user's abdominal skin rather than tug and pull at it to improve user's comfort. For example, the DN hydrogel may be configured to have an elastic modulus of about 9 kPa to about 10 kPa. Such DN hydrogel may conform to user's peristomal topography better and provide an improved adhesive seal when compared to hydrocolloid adhesives.

FIG. 3 is a schematic cross sectional view of an ostomy appliance 200 comprising an ostomy pouch 202 and an ostomy wafer 204 according to an embodiment. FIG. 4 is a schematic cross sectional view of the ostomy wafer 204. In an embodiment, the ostomy wafer 204 may comprise a backing layer 206 and a skin barrier 210 formed from the DN hydrogel of any of the foregoing embodiments. FIG. 5 is an illustration of an ostomy ring 300 formed from the DN hydrogel of any of the foregoing embodiments.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. An ostomy skin barrier formed from a double network hydrogel (DN hydrogel) comprising at least two different polymer networks.

2. The ostomy skin barrier of claim 1, wherein the DN hydrogel comprises a polyacrylamide network, a sodium alginate network, and a chitosan network, wherein the chitosan network interacts with user's skin when the ostomy skin barrier is attached to the user's skin to facilitate adhesion between the DN hydrogel and the user's skin.

3. The ostomy skin barrier of claim 2, wherein the DN hydrogel is formed from a mixture comprising sodium alginate, acrylamide (AAm), calcium sulphate (CaSO₄), ammonium persulfate (APS), N,N′-methylenebisacrylamide (MBAA), and tetramethylethylenediamine (TEMED).

4. The ostomy skin barrier of claim 3, wherein a mechanism for crosslinking the mixture is through a chemical means or a physical means, wherein the physical means is a light, temperature or mechanical stimulation.

5. The ostomy skin barrier of claim 1 4, wherein the DN hydrogel further comprises a pH biosensor.

6. The ostomy skin barrier of claim 5, wherein the pH biosensor is configured to change color when exposed to stoma dejecta to indicate a leakage.

7. The ostomy skin barrier of claim wherein the pH biosensor is a universal pH indicator configured to change color along a range of pH from about 1 to about 14, wherein the universal pH indicator is formed from a mixture of Methyl Red, Phenolphthalein, Ethanol, Bromothymol Blue, and NaOH.

8. The ostomy skin barrier of claim 5, wherein the pH biosensor is a pH indicator configured to change color along a range of pH from about 4 to about 10.

9. The ostomy skin barrier of claim 5, wherein the pH biosensor is configured to indicate a pH level of stoma dejecta that comes in contact with the ostomy skin barrier, wherein a color of the skin barrier is compared to a pH color scale for determination of the pH level of the stoma dejecta.

10. The ostomy skin barrier of claim 5, wherein the DN hydrogel is formed by adding the pH biosensor to an alginate/AAm solution prepared by dissolving sodium alginate and AAm in diH2O, and mixing MBAA, APS, CaSO4, and TEMED into the alginate/AAm solution to form a gel, and curing the gel using UV light.

11. The ostomy skin barrier of claim 1, wherein the DN hydrogel is configured to swell in response to a change in pH.

12. The ostomy skin barrier of claim 11, wherein the DN hydrogel is a basic DN hydrogel configured to swell when exposed to stoma dejecta having an acidic pH.

13. The ostomy skin barrier of claim 11, wherein the DN hydrogel is an acidic DN hydrogel configured to swell when exposed to stoma dejecta having a basic pH.

14. The ostomy skin barrier of claim 11, wherein the DN hydrogel is an amphiphilic hydrogel configured to swell when exposed to stoma dejecta having an acidic pH or a basic pH.

15. An ostomy appliance configured to secure an ostomy pouch to a user comprising the ostomy skin barrier according to claim 1.

16. The ostomy appliance of claim 15, wherein the ostomy appliance is an ostomy wafer attached to an ostomy pouch and configured to support the ostomy pouch when attached to the user.

17. The ostomy appliance of claim 15, wherein the ostomy appliance is an ostomy faceplate comprising a body-side coupling member, wherein the ostomy pouch includes a pouch-side coupling member configured to engage with the body-side coupling member to attach the ostomy pouch to the ostomy faceplate, wherein the ostomy faceplate is configured to support the ostomy pouch when attached to the user.

18. An ostomy ring formed from the ostomy skin barrier according to claim 1, wherein the ostomy ring is configured to work with an ostomy wafer or an ostomy faceplate to securely attach the ostomy wafer or the ostomy faceplate to a user.

19. The ostomy skin barrier of claim 1, wherein the DN hydrogel is configured to have viscoelastic properties similar to that of user's skin, wherein the DN hydrogel is configured to have an elastic modulus of about 10 kPa to about 1 MPa.
